(19) United States
(12) Patent Application Publication
(10) Pub. No.: US 2007/0124174 A1
(43) Pub. Date: May 31, 2007

(54) Medical alert jewelry

(76) Inventor: Libby Johnson, Huntersville, NC (US)

Correspondence Address: COX SMITH MATTHEWS INCORPORATED, 112 EAST PECAN STREET, SUITE 1800, SAN ANTONIO, TX, 78205-1521, US

(21) Appl. No.: 11/287,881

(22) Filed: Nov. 28, 2005

(51) Int. Cl. G06F 19/00; A61B 5/00

(52) U.S. Cl. 705/3

(57) ABSTRACT

A method for confidentially retaining and delivering a patient's medical information to emergency healthcare personnel through the use of stylish, luxurious, yet clearly identifiable medical alert jewelry. The jewelry contains information about the patient's specific medical condition as well as a toll-free telephone number where additional medical information may be accessed via a recorded message database.

### BACKGROUND OF THE INVENTION

1. Field of the Invention

The present invention is directed toward a method for confidentially retaining and delivering a patient's medical information to emergency healthcare personnel through the use of stylish, luxurious, yet clearly identifiable medical alert jewelry. In particular, the present invention informs emergency response personnel immediately that a medical condition exists while maintaining luxury and style. Furthermore, the jewelry contains information about the patient's specific medical condition as well as a toll-free telephone number where additional medical information may be accessed via a recorded message database.

2. Background Information

Every year over a hundred million people visit emergency rooms across the United States. Significantly, whether due to unconsciousness or some other inability to presently communicate, roughly ten million of these patients cannot effectively communicate their needs, associated medical problems, or medical history to emergency, healthcare personnel. This lack of communication, all too often leads to a patient either being misdiagnosed or given medication for which the patient has a history of adverse reactions. Many have attempted to address these problems associated with the inability of a patient to communicate with his or her emergency healthcare provider, not the least of which is medical alert jewelry. However, several problems exist with regard to presently available medical alert jewelry.

First, many patients refuse to wear the inexpensive medical jewelry available on the drug store shelves. Patients simply desire to wear something more attractive and fashionable than these over the counter type items. On the other hand, more attractive alternatives are available from fine jewelry makers which can run into the thousands of dollars. However, not only can this enterprise be expensive, emergency physicians also warn against wearing jewelry that is so fashionable that the medical insignia cannot be easily recognized by the appropriate medical emergency personnel. A balance between fashion and proper identification is lacking in currently available medical alert jewelry.

Another drawback to medical alert jewelry currently available is the trade-off one must make between medical confidentiality of the patient and the amount of detailed information provided to emergency healthcare personnel via the medical alert jewelry. Traditionally, the medical information one wishes to provide the emergency medical personnel must be engraved somewhere on the jewelry itself. This clearly presents two problems. First, space required to adequately present the needed, detailed, medical information is limited to the size of the surface area of the jewelry itself. Secondly, the patient must constantly wear their medical condition “on their sleeve” for anyone to access at any particular time. Each of these problems results in certain detailed medical information that may be vital to the emergency treatment of a patient either being written so small that it is difficult to read in an emergency or being left off of the medical alert jewelry altogether.

In view of the limitations of products and methods currently known in the art, a tremendous need exists for attractive yet identifiable medical alert jewelry capable of providing a significant amount of detailed medical information to emergency medical personnel while maintaining patient confidentiality. Applicant's invention, by its novel design provides a solution in view of currently available devices and methods.

### SUMMARY OF THE INVENTION

In view of the foregoing, it is an object of the present invention to provide attractive medical alert jewelry.

It is another object of the present invention to provide easily identifiable medical alert jewelry.

It is another object of the present device to provide a method of confidentially providing emergency medical personnel a patient's detailed medical information.

In satisfaction of these and other related objectives, the present invention provides medical alert jewelry that is attractive yet easily identifiable. The present invention also provides a method for confidentially retaining a patient's medical information for dissemination to emergency medical personnel as needed.

The present invention is packaged in attractive, stylish jewelry that tastefully yet clearly displays universally recognized medical alert insignia. This insignia quickly and efficiently informs emergency response personnel that a medical condition exists. The medical alert jewelry of the present invention further provides a compartment where medical information is stored rather than engraving on the outside of the jewelry itself in order to maintain both style and confidentiality. Once the jewelry compartment is opened, the name of the patient's specific medical condition and a toll-free telephone number is shown. Additionally, detailed medical information about the patient that has been confidentially collected is maintained in a database, which is easily accessed by medical personnel calling the toll-free number. This additional, detailed medical information is provided to medical personnel who dial the toll-free number by way of a pre-recorded message; thus, patient confidentiality is maintained without sacrificing the security of readily available emergency medical information.

### BRIEF DESCRIPTION OF THE DRAWINGS

N/A

### DETAILED DESCRIPTION OF THE PREFERRED EMBODIMENT

The present invention encompasses a method of delivering a patient's detailed medical information to emergency medical personnel through the combined use of stylish medical alert jewelry and a confidential patient database, which is accessed via telephone. In the preferred embodiment, the patient first selects a piece of attractive, stylish medical alert jewelry. The jewelry, although stylish and classy, prominently displays a universally recognizable medical insignia. The jewelry also contains a removable charm that is hidden from view during normal wearing of the medical alert jewelry yet easily accessed when needed.

Next, the patient, with the help of his or her physician, identifies all medical conditions and medical history that would be helpful to medical personnel in the event of the patient's medical emergency. From this information, the names of the pertinent conditions are printed in the “hidden” charm as well as a toll-free telephone number. The remainder of the pertinent medical history including details of any medical conditions and any known allergies to medication are confidentially recorded into and maintained by a voice-mail type database.

Finally, the patient merely wears his or her selected medical alert jewelry. In the case of a medical emergency, medical emergency personnel are able to quickly spot that the patient has some sort of medical condition by the universal medical alert symbol prominently displayed on the jewelry. The medical personnel may then remove the charm, which immediately reveals to them the pertinent medical conditions that exist with the patient as well as the toll-free telephone number. The medical professional may then call the toll-free number to gain additional, detailed information about the patient's medical conditions via the voice-mail database. Thus, not only does the present invention provide an attractive, more wearable alternative to existing medical alert jewelry, the present invention also delivers comprehensive medical information about the patient to medical personnel in the event of an emergency while maintaining the patient's confidentiality.

Although the invention has been described with reference to specific embodiments, this description is not meant to be construed in a limited sense. Various modifications of the disclosed embodiments, as well as alternative embodiments of the inventions will become apparent to persons skilled in the art upon reference to the description of the invention. It is, therefore, contemplated that the appended claims will cover such modifications that fall within the scope of the invention.

I claim:

1. A system for providing confidential medical information to emergency personnel, comprising:
an article of jewelry, said jewelry displaying medical alert insignia;
a charm, said charm being configured for printing a telephone number on said charm, said charm removably located within said article of jewelry such that said article of jewelry hides said telephone number from view until said charm is removed from said article of jewelry;
a database, said database being configured for recording a patient's medical information, said database configured for access via an emergency medical provider dialing said telephone number printed on said charm.

2. The system of claim 1 wherein said charm being further configured for printing the names of said patient's medical conditions on said charm.

3. A method for conveying confidential, medical information to emergency medical personnel, comprising the steps of:
recording a patient's medical information in a database, said database configured for access via telephone; and
printing a telephone number on an article of jewelry, said article of jewelry displaying medical alert insignia, said telephone number configured to provide access to said patient's medical information recorded in said database via telephone.

4. The method of claim 3 further comprising the step of printing the names of said patient's medical conditions on said article of jewelry.

5. A method for conveying confidential, medical information to emergency medical personnel, comprising the steps of:
recording a patient's medical information in a database, said database configured for access via telephone;
printing a telephone number on a charm., said telephone number configured to provide access to said patient's medical information recorded in said database via telephone; and
removably locating said charm inside an article of jewelry wherein said article of jewelry hides said telephone number from view until said charm is removed from said article of jewelry, said article of jewelry displaying a medical alert insignia.

6. The method of claim 5 further comprising the step of printing the names of said patient's medical conditions on said charm.
